(19) United States
(12) Patent Application Publication
(10) Pub. No.: US 2003/0040082 A1
(43) Pub. Date: Feb. 27, 2003

(54) Mutant pro-neurotrophin with improved activity

(76) Inventors: Mark Tuszynski, La Jolla, CA (US); Armin Blesch, San Diego, CA (US)

Correspondence Address: Stacy L. Taylor, FOLEY & LARDNER, 402 West Broadway, 23rd Floor, San Diego, CA, 92101-3542, US

(21) Appl. No.: 09/788,188

(22) Filed: Feb. 16, 2001

(51) Int. Cl. A61K 48/00; C07H 21/04; C12N 5/06; A61K 38/18; C07K 14/48

(52) U.S. Cl. 435/69.4; 424/93.21; 435/325; 536/23.5; 530/399; 435/320.1

(57) ABSTRACT

Mutant “pro-neurotrophins” whose corresponding growth factor is secreted more efficiently from host cells than wild-type growth factor. Such improved activity is obtained through substitution of a residue in the precursor protein (“prepro”) region of a pro-form of a growth factor. Pro-neurotrophins contain at least one conserved asparagine-based N-glycosylation site present upstream of the cleavage site for separation of the corresponding neurotrophin. The invention substitutes the asparagine with a basic residue, such as serine. At the higher levels of extracellular growth factor achieved by the invention, the bioavailability, and therefore the therapeutic potential of the corresponding mature protein is enhanced.

### BACKGROUND OF THE INVENTION

1. Field of the Invention

The invention relates to a mammalian nervous system growth factors (“neurotrophic factors”).

2. History of the Related Art

Nervous system growth factors consist of several families of proteins which play a physiological role in the development and regulation of neurons in mammals. In adults, several populations of neurons, including basal forebrain cholinergic neurons, motor neurons and sensory neurons, retain responsiveness to neurotrophic factors and in their presence can regenerate after loss or damage. For this reason, growth factors are considered to have great promise for the treatment of neurodegenerative conditions such as Alzheimer's Disease (AD), Parkinson's Disease (PD), amyotrophic lateral sclerosis (ALS), multiple sclerosis, stroke, peripheral sensory neuropathies and spinal cord injuries.

The family of “neurotrophins” includes nerve growth factor NGF (see, U.S. Pat. No. 5,169,762); brain-derived neurotrophic factor (BDNF), also referred to as neurotrophin-2 (Leibrock et al. (Nature, 341: 49-152 [1989]), and neurotrophin-3. (Emfors et al., Proc. Natl. Acad. Sci. USA, 87:5454-5458 [1990]; Hohn et al., Nature, 344:339 [1990]; Maisonpierre et al., Science, 247:1446 [1990]; Rosenthal et al., Neuron, 4:767 [1990]; Jones and Reichardt, Proc. Natl. Acad. Sci. USA, 87:8060-8064 [1990]; Kaisho et al., FEBS Lett., 266:187 [1990]). Neurotrophin-4/5 (referred to as either NT4 or NT5) has been identified (Hallbook et al., Neuron, 6:845-858 [1991]; Berkmeier et al., Neuron, 7:857-866 [1991]; Ip et al., Proc. Natl. Acad. Sci. USA, 89: 3060-3064 [1992]). U.S. Pat. No. 5,364,769, issued Nov. 15, 1994, discloses human NT-4/5 and processes for its recombinant expression and is incorporated herein by reference. Also reported are chimeric and pantropic neurotrophins, such as that reported in U.S. Pat. No. 5,488,099, issued Jan. 30, 1996, in Urfer et al., EMBO J. 13(24):5896-909 (1994).

Other nervous system growth factors include neurotrophin-6 (NT-6), ciliary neurotrophic factor (CNTF), glial cell line-derived neurotrophic factor (GDNF), the fibroblast growth factor family (FGF's 1-15), leukemia inhibitory factor (LIF), certain members of the insulin-like growth factor family (e.g., IGF-1), the neurturins, persephin, the bone morphogenic proteins (BMPs), the immunophilins, the transforming growth factor (TGF) family of growth factors, the neuregulins, epidermal growth factor (EGF), platelet-derived growth factor (PDGF), and others. NGF and NT-3 in particular have been tested with promising results in animal studies (see, e.g., Hefti and Weiner, Ann Neurol., 20:275-281 (1986); Tuszynski and Gage, Ann. Neurol., 30:625-636 (1991); Tuszynski, et al., Gene Therapy, 3:305-314 (1996) and Blesch and Tuszynski, Clin.Neurosci., 3:268-274 (1996)).

Most nervous system growth factors are initially synthesized as precursors containing an amino-terminal propeptide that is proteolytically processed to release the mature growth factor. Because growth factors are normally expressed at low levels, little is known about how they are sorted and secreted in vivo by neurons and other cell types.

There is a significant degree of structural similarity among the neurotrophins and pro-neurotrophin polypeptides. For example, NGF, NT4/5, BDNF, NT-6 and NT-3 are all generated from precursors of comparable size which contain, at their N-termini, hydrophobic signal peptides followed by pro-regions containing sequences of contiguous basic amino acids. The presumptive signal sequence, the cysteine residues important for tertiary structure, possible glycosylation sites, and dibasic amino acids required for proteolytic cleavage to mature neurotrophin are also conserved among species (Whittemore, et al., J Neurosci Res., 20:403-10, 1988, incorporated herein by this reference). One glycosylation site, conserved throughout the family, is located in the prepro region of pro-neurotrophins, upstream of the cleavage site for the mature protein. In general, intracellular cleavage of pro-neurotrophins to produce active nerve growth factors takes place following the precursor motif Arg-Xaa-(Lys/Arg)-Arg, where Xaa=Ser in proNGF and proNT4/5; Xaa=Val for proBDNF and proNT-6 and Xaa=Arg for proNT-3.

Pro-neurotrophin coding sequences share a remarkable degree of homology, both with each other and among species (e.g., coding for mouse NGF is very similar to coding for human NGF). A number of such sequences have been reported; e.g., a cDNA including the coding sequence for hNGF is reported in GenBank at E03015 (Kazuo, et al., Japanese Patent Application No. JP19911175976-A; the nucleotide sequence of genomic hNGF (with putative amino acid sequence) is reported in GenBank at V01511 (Ullrich, Nature, 303:821-825 (1983); see also, SEQ.ID.Nos.1-2); and, the genomic nucleotide sequence of hNT-3 is reported in GenBank at E07844 (Asae, et al., JP Patent Application No. 1993189770-A4). For ease of reference, a chart comparing the BDNF, hNGF and NT-3 coding sequences is provided in FIG. 2; precursor coding and amino acid sequences for these neurotrophins, as well as NT-4/5, are also set forth herein for reference (as, respectively, SEQ.ID.Nos. 3-6).

Despite many of their structural similarities, nervous system growth factors act on discrete and different targets. Little is known about which amino acid residues within a growth factor are necessary to its activity. It has been reported that elimination of as little as the first 9 residues of the N-terminus and the last two residues from the C-terminus of purified recombinant human NGF produces a neurotrophin molecule which is 300-fold less efficient in binding activity as compared to wild-type hNGF, and 50- to 100-fold less efficient at stimulating dorsal root ganglion and sympathetic ganglion survival (U.S. Pat. No. 6,090,781).

Still, because of their potential therapeutic significance, efforts continue toward improvement of the bioavailability of nervous system growth factor polypeptides in vivo. Most such efforts have focused on fusion of a signal peptide, such as the E. coli OmpA signal peptide, to a synthetic or recombinant nerve growth factor (see, e.g., U.S. Pat. Nos. 4,757,013; 4,338,397; and 5,608,036). Other approaches alter the tertiary structure of the nerve growth factor protein (U.S. Pat. No. 5,235,043). Such modified proteins are reportedly secreted more efficiently from a host cell than are unaltered nerve growth factors, but require use of a protein which differs in size or structure from the native protein, at the risk of adversely affecting binding specificity and protein stability.

### SUMMARY OF THE INVENTION

The invention provides mutant nervous system growth factors with improved activity. In particular, the pro-neurotrophins of the invention enable a mature neurotrophin protein to be secreted more efficiently from host cells than wild-type neurotrophins. At the higher levels of extracellular growth factor achieved by the invention, the bioavailability, and therefore the therapeutic potential of the mature protein is enhanced.

Such improved activity is obtained through substitution of a residue in the precursor protein (“prepro”) region of a pro-neurotrophin. Pro-neurotrophins contain at least one N-glycosylation sequence which is completely conserved throughout the family. The invention targets at least one of these sequences. In particular, the substitution is of a basic residue for an asparagine.

It is notable that previous studies of the role of N-glycosylation of NGF in secretion regulation have concluded that certain mutations to the site targeted in the invention reduces secretion of the mature polypeptide (Heymach, et al., J.Biol. Chem., 271:25430-25437 (1996)). As such, the increase in secretion obtained through practice of the invention is especially surprising.

Synthetic and recombinant forms of the improved nervous system growth factors of the invention are provided, as are methods for their preparation and pharmaceutical compositions for their use in vivo.

### DESCRIPTION OF THE PREFERRED EMBODIMENTS

### I. Pro-neurotrophins of the Invention

“Pro-neurotrophin” refers to a molecule consisting of a precursor protein joined by a cleavage site to a corresponding growth factor. The corresponding growth factor may be of any family, and is preferably of the NGF family, including the neurotrophins (NGF, NT-3, NT-4/5 and BDNF). The growth factor may be derived from any species, including murine, bovine, ovine, porcine, equine, avian, and preferably human, in native sequence or in a genetically engineered form, and from any source, whether natural, synthetic, or recombinantly produced. Preferred for therapeutic use in humans is a mutant human pro-nervous system growth factor prepared in accord with the invention.

“Mutant pro-neurotrophins” of the invention are produced through substitution of a basic residue, such as serine or a similarly charged residue, for the asparagine of a targeted N-glycosylation site. In neurotrophin family, the targeted N-glycosylation site is located 4 or 8 amino acids upstream of the cleavage site for the corresponding neurotrophin. This site is conserved in all known neurotrophin-family pro-neurotrophins. As such, it is reasonably expected that substitutions made according to the invention to all neurotrophin-family pro-neurotrophins will produce comparable improvements in secretion efficiency.

The amino acid sequence of wild-type human NGF pro-neurotrophin (GenBank P01138; SEQ.ID.No.1) is below. The N-glycosylation sites are indicated by underlining, and the cleavage site is indicated by an underlined space. The targeted N-glycosylation site is indicated in large type; it is at position 114, 8 amino acids upstream of the start of the mature protein, at position 122. This site is present in the prepro region of all neurotrophin-family pro-neurotrophins.

SEQ.ID.No. 1:

| | |
|---|---|
| 1 | MSMLFYTLIT AFLIGIQAEP HSESNVPAGH TIPQVHWTKL QHSLDTALRR ARSAPAAAIA |
| 61 | ARVAGQTRNI TVDPRLFKKR RLRSPRVLFS TQPPREAADT QDLDFEVGGA APFNRTHRSK |
| 121 | R_SSSHPIFHR GEFSVCDSVS VWVGDKTTAT DIKGKEVMVL GEVNINNSVF KQYFFETKCR |
| 181 | DPNPVDSGCR GIDSKHWNSY CTTTHTFVKA LTMDGKQAAW RFIRIDTACV CVLSRKAVRR |
| 241 | A |

SEQ.ID.No. 2 is a mutant NGF pro-neurotrophin, wherein the asparagine in the targeted N-glycosylation site is replaced by a serine.

The amino acid sequence of wild-type human BDNF pro-neurotrophin (GenBank P23560; SEQ.ID.No.3) is below. The cleavage site is indicated by an underlined space. The targeted N-glycosylation site is indicated in large type; it is at position 121, 8 amino acids upstream of the start of the mature protein, at position 129.

| | |
|---|---|
| 1 | MTILFLTMVI SYFGCMKAAP MKEANIRGQG GLAYPGVRTH GTLESVNGPK AGSRGLTSLA |
| 61 | DTFEHVIEEL LDEDQKVRPN EENNKDADLY TSRVMLSSQV PLEPPLLFLL EEYKNYLDAA |
| 121 | NMSMRVRR HS DPARRGELSV CDSISEWVTA ADKKTAVDMS GGTVTVLEKV PVSKGQLKQY |
| 181 | FYETKCNPMG YTKEGCRGID KRHWNSQCRT TQSYVRALTM DSKKRIGWRF IRIDTSCVCT |
| 241 | LTIKRGR |

SEQ.ID.No. 4 is a mutant BDNF pro-neurotrophin, wherein the asparagine in the targeted N-glycosylation site is replaced by a serine.

The amino acid sequence of wild-type human NT-3 pro-neurotrophin (GenBank P20783; SEQ.ID.No. 5) is below. The cleavage site is indicated by an underlined space. The targeted N-glycosylation site is indicated in large type; it is at position 131, 8 amino acids upstream of the start of the mature protein, at position 139.

| | |
|---|---|
| 1 | MSILFYVIFL AYLRGIQGNN MDQRSLPEDS LNSLIIKLIQ ADILKNKLSK QMVDVKENYQ |
| 61 | STLPKAEAPR EPERGGPAKS AFQPVIAMDT ELLRQQRRYN SPRVLLSDST PLEPPPLYLM |
| 121 | EDYVGSPVVA NRTSRRKR YA EHKSHRGEYS VCDSESLWVT DKSSAIDIRG HQVTVLGEIK |
| 181 | TGNSPVKQYF YETRCKEARP VKNGCRGIDD KHWNSQCKTS QTYVRALTSE NNKLVGWRWI |
| 241 | RIDTSCVCAL SRKIGRT |

SEQ.ID.No. 6 is a mutant NGF pro-neurotrophin, wherein the asparagine in the targeted N-glycosylation site is replaced by a serine.

The amino acid sequence of wild-type human NT-4/5 pro-neurotrophin (GenBank P NP 006170; SEQ.ID.No. 7) is below. The cleavage site is indicated by an underlined space. The targeted N-glycosylation site is indicated in large type; it is at position 76, 4 amino acids upstream of the start of the mature protein, at position 81.

| | |
|---|---|
| 1 | MLPLPSCSLP ILLLFLLPSV PIESQPPPST LPPFLAPEWD LLSPRVVLSR GAPAGPPLF |
| 61 | LLEAGAFRES AGAPANRSRR GVSETAPASR RGELAVCDAV SGWVTDRRTA VDLRGREVEV |
| 121 | LGEVPAAGGS PLRQYFFETR CKADNAEEGG PGAGGGGCRG VDRRHWVSEC KAKQSYVRAL |
| 181 | TADAQGRVGW RWIRIDTACV CTLLSRTGRA |

SEQ.ID.No. 8 is a mutant NT-4/5 pro-neurotrophin, wherein the asparagine in the targeted N-glycosylation site is replaced by a serine.

For use in preparing recombinant mutant pro-neurotrophins, precursor coding sequences for pro-neurotrophins are known and publicly available through database services such as GenBank. For ease of reference, reported precursor coding sequences for NGF, NT-3, NT 4/5 and BDNF include:

```
SEQ.ID.No. 9 [GenBank V01511]:
Coding sequence for NGF, including prepro region
(target nucleotide is capitalized in bold type)
ATGTCCATGTTGTTCTACACTCTGATCACAGCTTTTCTGATCGGCATACAGGCGGAACCACACT
CAGAGAGCAATGTCCC
TGCAGGACACACCATCCCCCAAGTCCACTGGACTAAACTTCAGCATTCCCTTGACACTGCCCTT
CGCAGAGCCCGCAGCG
CCCCGGCAGCGGCGATAGCTGCACGCGTGGCGGGGCAGACCCGCAACATTACTGTGGACCCC
AGGCTGTTTAAAAAGCGG
CGACTCCGTTCACCCCGTGTGCTGTTTAGCACCCAGCCTCCCCGTGAAGCTGCAGACACTCAGG
ATCTGGACTTCGAGGT
CGGTGGTGCTGCCCCCTTCAACAGGACTCACAGGAGCAAGCGGTCATCATCCCATCCCATCTT
CCACAGGGGCGAATTCT
CGGTGTGTGACAGTGTCAGCGTGTGGGTTGGGGATAAGACCACCGCCACAGACATCAAGGGC
AAGGAGGTGATGGTGTTG
GGAGAGGTGAACATTAACAACAGTGTATTCAAACAGTACTTTTTTGAGACCAAGTGCCGGGAC
CCAAATCCCGTTGACAG
CGGGTGCCGGGGCATTGACTCAAAGCACTGGAACTCATATTGTACCACGACTCACACCTTTGT
CAAGGCGCTGACCATGG
ATGGCAAGCAGGCTGCCTGGCGGTTTATCCGGATAGATACGGCCTGTGTGTGTGTGCTCAGCA
GGAAGGCTGTGAGAAGA
GCCTGA
SEQ.ID.No. 10 [GenBank BF446664]:
Coding sequence for NT-3 precursor
                TTTGAAGGCAAGCACACTGATTTTATTGAGAAAAAAGCTTATATACTGTAGGGTTGCTGA
AGTTTAATAAATAAAGGTCAACTTATAATATATAAAAACAATATAAACATTTATATGCTA
CATGCATATCATATAATTTAAAGTAATAATTTATATATGGGGAGAGATGCCAATTCATGT
TCTTCCGATTTTTCTCGACAAGGCACACACACAGGACGTGTCTATCCGTATCCACCGCCA
GCCCACGAGTTTATTGTTCTCTGAAGTCAGTGCTCGGACGTAGGGTTGGGATGTTTTGCA
CTGAGAGTTCCAGTGTTTATCATCAATACCCCTGCAACCGTTTTTGACCGGCCTGGCTTC
CTTACATCGCGTTTCATAAAAATATTGTTTGACAGGAGAGTTGCCCGTTTTGATCTCCCC
CAGCACCGTGACCTGGTGTCCCCGAATGTCGATGGCCGATGACTTGCGGTCACCCACAGA
CTCTCACTGTCACATACCGAGTACTCCCCTCGGTGACTCTTATGNCTCGCGTACCGTTTT
CCGCCGGATGTTCTGTTCGCCACCACGGGGCTTGCCACGTAATCCTCCATGAGATACAAG
GGCGGNGGCTCCCAAGGGNNTGTGTCGCTCANCAGGAACCCGNGTGAGTGTAGCGGCTCT
GTTGTCGCAGAAGTTC
SEQ.ID.NO. 11 [GenBank AI595076]:
coding sequence for NT-4 precursor
  CTGGATTCGGATCGACACAGCTTGCGTCTGCACGCTCCTCAGCCGAACAGGCCGAGCCTG
AGGTCCAGGCTTGGGAACTGCCCAAGTTGAGGGAAAACAAAAAACAAAAAACCAAAGCTG
GATGCTGAAAGGACCACAGGGGTGGCCTGGCTGCTCTACCGTGCCTTATGACTGGGAACT
GGAATAACCAAAGAATCAAATCTCTCTCAAATCTCAGTCTGTGTGGAATGTATGGTGAAA
CCAAATGAGGTTTCAAGTGATGAATAGGAGTTCTCCCGGAGGAACTTGACATTAATAACA
ATAGCCAATGTTTACTATCTCCTGTTTATCAGACCTGATATATGACTTTGGCAACCATTT
TAACATTCAGAGACCCTGGCTCATCAAAACGGACGAGGAAAGAACGCATGAAAAGGGGAT
GCATGATGCATGCGCTGGAGCTAGGCCTCCATCAGTAGGCTGTTTCTG
SEQ.ID.NO. 12 [GenBank NM 006179]:
coding sequence for NT-4/5
(precursor at nucleotides 73-240)
    ATGCTCCCTC TCCCCTCATG CTCCCTCCCC ATCCTCCTCC TTTTCCTCCT CCCCAGTGTG
    CCAATTGAGT CCCAACCCCC ACCCTCAACA TTGCCCCCTT TTCTGGCCCC TGAGTGGGAC
    CTTCTCTCCC CCCGAGTAGT CCTGTCTAGG GGTGCCCCTG CTGGGCCCCC TCTGCTCTTC
    CTGCTGGAGG CTGGGGCCTT TCGGGAGTCA GCAGGTGCCC CGGCCAACCG
CAGCCGGCGT
    GGGGTGAGCG AAACTGCACC AGCGAGTCGT CGGGGTGAGC TGGCTGTGTG
CGATGCAGTC
    AGTGGCTGGG TGACAGACCG CCGGACCGCT GTGGACTIGC GTGGGCGCGA
GGTGGAGGTG
    TTGGGCGAGG TGCCTGCAGC TGGCGGCACT CCCCTCCGCC AGTACTTCTT
TGAAACCCGC
    TGCAAGGCTG ATAACGCTGA GGAAGGTGGC CCGGGGGCAG GTGGAGGGGG
CTGCCGGGGA
    GTGGACAGGA GGCACTGGGT ATCTGAGTGC AAGGCCAAGC AGTCCTATGT
GCGGGCATTG
    ACCGCTGATG CCCAGGGCCG TGTGGGCTGG CGATGGATTC GAATTGACAC
TGCCTCGCTC
    TGCACACTCC TCAGCCGGAC TGGCCGGGCC TGA
SEQ.ID.NO. 13 [GenBank BF439589]:
coding sequence for BDNF precursor
  TTTTTATTTTTTTTAACTTTTTATGTTTTCAGTTCTTGGCAACGGCAACAAACCACAACA
TTATCAAGGAATGTAATGCAGACTTTTTAAGTTGTGCGCAAATGACTGTTTCCCTTCTGG
TCATGGACATGTCCAATAAATAGATTGTAGAACCACTGTACTGTATAAACTTCATTTATA
CATGCAGTTCATAAAATTATTTTTTTCTTAACTGAATAATTTACCCTGGTATGTATATAT
TACAAATAGATAATTTTTGTCTCAATATAATCTAATCTATACAACATAAATCCACTATCT
TCCCCTTTTAATGGTCAATRTACATACACAAGAAGTGTCTATCCTTATGAATCGCCAGCC
AATTCTCTTTTTGCTATCCATGGTAAGGGCCCGCACGTACGACTGGGTAGTTCGGCACTG
GGAGTTCCAATGCCTTTTGTCTATGCCCCTGCAGCCTTCTTTTGT
```

The following discussion refers to a mutant NGF pro-neurotrophin to illustrate making of the desired mutations of the invention, although the invention will be understood not to be limited to modification of NGF pro-neurotrophins.

The targeted N-linked glycosylation site in the NGF prepro region consists of an asparagine 8 amino acids upstream from the cleavage site of the mature protein. Thus, the described mutation lies within the prepro sequence of NGF, but at a distance from the cleavage site sufficient to avoid interference with processing of the mature peptide.

The mutation was introduced by amplification of the wild-type sequence using polymerase chain reaction, using the following primers:

Forward primer containing a HpaI restriction site and Kozak consensus sequence:

TGT GTTAAC GCCACC ATG TCC ATG TTG TTC TAC ACT (SEQ.ID.No. 14)

Reverse primer containing a BamHI site:

TGT GGATCC TCA GGC TCT TCT CAC AGC CTT (SEQ.ID.No. 15)

The PCR product was digested with HpaI and BamHI and cloned into the retroviral vector pLXSN. Plasmid DNA was isolated from E.coli and the insert sequenced from both sides. The base pair exchange thereby achieved alters position 8976 of the NGF coding sequence as shown in SEQ.ID.No. 1 to change the wild-type codon AAC coding for acidic residue asparagine to a mutated codon AGC coding for the basic residue serine, as follows:

ATGTCCATGTTGTTCTACACTCTGATCACAGCTTTTCTGATCGGCATACAGGCGGAACCACACT
CAGAGAGCAATGTCCC
TGCAGGACACACCATCCCCCAAGTCCACTGGACTAAACTTCAGCATTCCCTTGACACTGCCCTT
CGCAGAGCCCGCAGCG
CCCCGGCAGCGGCGATAGCTGCACGCGTGGCGGGGCAGACCCGCAACATTACTGTGGACCCC
AGGCTGTTTAAAAAGCGG
CGACTCCGTTCACCCCGTGTGCTGTTTAGCACCCAGCCTCCCCGTGAAGCTGCAGACACTCAGG
ATCTGGACTTCGAGGT
CGGTGGTGCTGCCCCCTTCAgCAGGACTCACAGGAGCAAGCGGTCATCATCCCATCCCATCTT
CCACAGGGGCGAATTCT
CGGTGTGTGACAGTGTCAGCGTGTGGGTTGGGGATAAGACCACCGCCACAGACATCAAGGGC
AAGGAGGTGATGGTGTTG
GGAGAGGTGAACATTAACAACAGTGTATTCAAACAGTACTTTTTTGAGACCAAGTGCCGGGAG
CCAAATCCCGTTGACAG
CGGGTGCCGGGGCATTGACTCAAAGCACTGGAACTCATATTGTACCACGACTCACACCTTTGT
CAAGGCGCTGACCATGG
ATGGCAAGCAGGCTGCCTGGCGGTTTATCCGGATAGATACGGCCTGTGTGTGTGTGCTCAGCA
GGAAGGCTGTGAGAAGA
GCCTGA
(SEQ.ID.No. 16; the mutation is noted by underlining and bold type)

To evaluate the effect of this mutation on the activity of the mature NGF protein product, primary non-human primate fibroblasts were established as in vitro cell cultures and the wild-type or mutant NGF genes were introduced into the cells using MLV expression vectors. After stable infection was established by neomycin selection, supernatants from the cell cultures were sampled for a 24 hour period to detect NGF production. The following results were obtained:

NGF from mutated pro-neurotrophin: 28.7±5.6 ng/10⁶ cells/day

NGF from wild-type pro-neurotrophin: 13.2±1.7 ng/10⁶ cells/day

These differences are significant (p-value=0.038 by Student's t-test); and represent a substantial improvement of the secretion efficiency of mature HNGF, achieved through a minimal structural alteration of an NGF pro-neurotrophin. Although not limiting of the scope of the invention, a likely mechanism of action responsible for the results achieved is interference with natural glycosylation of the prepro protein, given that the substitutions made according to the invention lie within a conserved N-linked glycosylation site.

### II. Methods for Production of Mutant Pro-neurotrophins

Mutant pro-neurotrophins are prepared according to the invention by chemical synthesis (with reference to the wild-type peptide sequences set forth in SEQ.ID.Nos. 1, 3, 5 and 7) or, preferably, through expression as a recombinant peptide (with reference to the wild-type coding sequences set forth in SEQ.ID.Nos. 9-13). Techniques for production of polypeptides according to each of these methods are well-known in the art and so will only be described briefly here.

Recombinant pro-neurotrophins can be produced in vitro or in vivo through expression of a coding sequence for the pro-neurotrophin protein, including the prepro region (e.g., SEQ.ID.Nos. 9-13). Site-directed mutagenesis techniques, including those accomplished through PCR as described in the preceding section, are widely known in the art and can be applied by one of ordinary skill in the art to accomplish the mutations required by the invention.

In general, prokaryotes are used for cloning of DNA sequences in constructing recombinant expression vectors. For example, E. coli K12 strain 294 (ATCC Accession No. 31446) may be particularly useful. Prokaryotes also are used for expression. The aforementioned strain, as well as E. coli W3110 (ATTC Accession No. 27325), bacilli such as Bacillus subtilus, and other enterobacteriaceae such as Salmonella typhimurium or Serratia marcescans, and various pseudomonas species may also be used for expression.

Non-viral plasmid vectors which may be used in the invention contain promoters and control sequences which are derived from species compatible with the host cell. The vector ordinarily carries a replication site as well as marker sequences which are capable of providing phenotypic selection in transformed cells. For example, E. coli is typically transformed using pBR322, a plasmid derived from an E. coli species (Bolivar, et al., Gene, 2:95, 1977). pBR322 contains genes for ampicillin and tetracycline resistance and thus provides easy means for identifying transformed cells. The pBR322 plasmid, or other microbial plasmid must also contain or be modified to contain promoters and other control elements commonly used in recombinant DNA construction.

Expression may also be achieved using RNA or DNA viruses, including retroviruses, adenoviruses, herpes virus, vaccinia and adeno-associated viruses. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), human immunodeficiency virus (HIV-1) and Rous Sarcoma Virus (RSV). Adeno-associated viruses are especially useful for their stable expression and relative lack of adverse side effects when delivered in vivo.

A number of viral vectors can incorporate multiple genes. All of these vectors can transfer or incorporate a gene for a selectable marker so that transduced cells can be identified and generated. By inserting one or more sequences of interest into the viral vector, along with another gene which encodes the ligand for a receptor on a specific target cell, for example, the vector is now target specific.

Further, in addition to prokaryotes, eukaryotic microbes such as yeast cultures may also be used. Saccharomyces cerevisiae, or common baker's yeast is the most commonly used eukaryotic microorganism for use in in vitro expression of polynucleotides, although a number of other strains are commonly available.

Suitable promoting sequences for use with yeast hosts include the promoters for 3-phosphoglycerate kinase (Hitzeman, et al., J. Biol. Chem., 255:2073, 1980) or other glycolytic enzymes (Hess, et al. J. Adv. Enzyme Reg. 7:149, 1968; and Holland, Biochemistry, 17:4900, 1978) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degraded enzymes associated with nitrogen metabolism, metallothionine, glyceraldehyde-3-phosphate dehydrogenase, and enzymes responsible for maltose and galactose utilization. Yeast enhancers also are advantageously used with yeast promoters.

Pro-neurotrophins can also be readily synthesized by conventional techniques, such as the solid phase synthesis techniques as described in Gutierrez, et al., FEBS Letters, 372:39-43 (1995), the disclosure of which is incorporated herein by this reference to illustrate knowledge in the art concerning techniques for the production of synthetic peptides.

Briefly, commonly used methods such as t-Boc or Fmoc protection of alpha-amino groups are suitable for use in synthesizing pro-neurotrophins of the invention. Both methods involve stepwise syntheses whereby a single amino acid is added at each step starting from the C terminus of the peptide (see, Coligan, et al., Current Protocols in Immunology, Wiley Interscience, 1991, Unit 9). In this fashion, the desired substitution of a basic residue for an acidic one would be made during the synthesis process.

Pro-neurotrophins of the invention can also be synthesized by various well known solid phase peptide synthesis methods, such as those described by Merrifield (J. Am. Chem. Soc., 85:2149, 1962), and Stewart and Young (Solid Phase Peptides Synthesis, Freeman, San Francisco, 1969, pp 27-62), using a copoly (styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at OEC. After evaporation of the reagents, the peptides are extracted from the polymer with 1% acetic acid solution which is then lyophilized to yield the crude material. This can normally be purified by such techniques as gel filtration on a “SEPHADEX G-15” or “SEPHAROSE” affinity column. Lyophilization of appropriate fractions of the column will yield the homogeneous peptide or peptide derivatives, which can then be characterized by such standard techniques as amino acid analysis, thin layer chromatography, high performance liquid chromatography (HPLC), ultraviolet absorption spectroscopy, molar rotation, solubility, and quantitated by the solid phase Edman degradation.

In order to further increase the bioavailability of pro-neurotrophins prepared according to the invention, the pro-neurotrophins may be synthesized using standard Fmoc or t-Boc chemistries with amino acid derivatives in D-conformation. Alternatively, sequences with reduced peptide bonds in positions susceptible to proteolysis may be synthesized according to, for example, Meyer et al., J.Med.Chem., 38:3462-3468 (1995) (incorporated herein for reference). Briefly, such peptides are synthesized using a Fmoc/tert-butyl strategy, and the Y(CH₂NH) bonds, or reduced bonds, are introduced via reductive alkylation of the N-terminal amino group of the growing peptide with a Fmoc-Na-protected amino aldehyde.

D-isomers of pro-neurotrophins are also desirable for their resistance to proteolytic degradation in vivo. It is well recognized that L-bond peptides are susceptible to proteolytic degradation, restricting their application as drugs. However, this obstacle has been successfully bypassed in some cases by synthesizing analogues which contain D-bond amino acids or non-natural amino acids. The addition of a single D-amino acid at the C-terminal position is enough to enhance the resistance to proteolytic degradation by exopeptidases, without significantly altering the secondary structure of the peptide (Abiko, supra). Resistance to endopeptidases can be achieved by including individual non-cleavable non-peptidic bonds in points in the peptide sequence that are specially sensitive to enzymatic degradation (Meyer, et al., J. Med. Chem. 38:3462-3468 (1995); Guichard, et al., Peptide Research 7:308-321 (1994)). Reverse amide bonds Y[NHCO], reduced amide bonds Y[CH₂NH] or retro-reduced bonds Y[NHCH₂] can be used as surrogates of the amide link [CONH] in ESUPs of the invention. Reduced amide links are preferred, since they result only in minor destabilization of a-helices (Dauber-Osguthorpe, et al., Int. J. Pep. Prot. Res. 38:357-377 (1991).

Alternatively, pro-neurotrophins can be synthesized in an all-D-conformation. D-peptides can be equally active as the original all-L-peptides (Merrifield, et al., Ciba Foundation Symposium 186:5-20 (1994); Wade, et al., Proc. Natl. Acad. Sci. USA 87:4761-4765 (1990)), capable of successfully resisting enzymatic degradation and less immunogenic than their all-L-analogues (King, et al., J. Immunol. 153:1124-1131 (1994)).

### III. Pharmaceutical Compositions of Pro-neurotrophins and Pro-neurotrophin Coding Polynucleotides

Pharmaceutically useful compositions of pro-neurotrophins are prepared by mixing the peptide with physiologically acceptable carriers. Such carriers will be nontoxic to recipients at the dosages and concentrations employed. Ordinarily, the preparation of such compositions entails combining the particular protein with buffers, antioxidants such as ascorbic acid, low molecular weight (less than about 10 residues) polypeptides, proteins, amino acids, carbohydrates including glucose or dextrins, chelating agents such as EDTA, glutathione and other stabilizers and excipients.

Such compositions may be lyophilized for storage and will be reconstituted according to pharmaceutically acceptable means; i.e., suitably prepared and approved for use in the desired application. A sodium chloride free buffer is preferred for use as a reconstituting agent. Whatever its form, the composition product will be placed into sterile containers (e.g., ampules) for storage and transportation.

A therapeutic kit product may also include sterile saline or another pharmaceutically acceptable emulsion and suspension base for use in reconstituting lyophilized pro-neurotrophin suspensions, suitably labeled and approved containers of pro-neurotrophin compositions. Such a kit may also comprise a carrier means being compartmentalized to receive in close confinement one or more container means such as vials, tubes, and the like, each of the container means comprising one of the separate elements to be used in a therapeutic application.

Examples illustrating the construction and use of the invention are provided below. These examples do not limit the scope of the invention, which is defined by the appended claims. Standard abbreviations (e.g., “ml” for milliliters) are used in the examples unless otherwise noted.

What is claimed is:

1. A mutant pro-neurotrophin for use in intracellular processing of a corresponding growth factor having improved secretion efficiency as compared to a wild-type growth factor, wherein the wild-type pro-neurotrophin has an asparagine residue at a position 8 amino acids upstream from the site of cleavage for the mature growth factor, the mutant pro-neurotrophin comprising a polypeptide in which the wild-type asparagine residue is replaced by a basic residue.

2. The mutant pro-neurotrophin according to claim 1, wherein the basic residue is serine.

3. The mutant pro-neurotrophin according to claim 1, wherein the corresponding growth factor is selected from the group consisting of neurotrophins NGF, NT-3 and BDNF.

4. The mutant pro-neurotrophin according to claim 1, wherein the polypeptide is a recombinant one, and the replacement of the wild-type asparagine is made by mutation of a polynucleotide encoding the wild-type pro-neurotrophin.

5. A mutant pro-neurotrophin for use in intracellular processing of a corresponding growth factor having improved secretion efficiency as compared to a wild-type growth factor, wherein the wild-type pro-neurotrophin has an asparagine residue at a position 4 amino acids upstream from the site of cleavage for the mature growth factor, the mutant pro-neurotrophin comprising a polypeptide in which the wild-type asparagine residue is replaced by a basic residue.

6. The mutant pro-neurotrophin according to claim 5, wherein the basic residue is serine.

7. The mutant pro-neurotrophin according to claim 5, wherein the corresponding neurotrophin is NT-4/5.

8. The mutant pro-neurotrophin according to claim 5, wherein the polypeptide is a recombinant one, and the replacement of the wild-type asparagine is made by mutation of a polynucleotide encoding the wild-type pro-neurotrophin.

9. A mutant pro-neurotrophin precursor polypeptide selected from the group of polypeptides consisting of SEQ.ID.Nos. 1, 3, 5 and 7.

10. A mutant pro-neurotrophin comprising the precursor polypeptide of claim 5, joined by a cleavage site to a corresponding mature growth factor.

11. A polynucleotide encoding a mutant pro-neurotrophin, wherein the polynucleotide differs in nucleotide sequence from wild-type by replacement of the codon encoding a target asparagine residue, at a position 8 amino acids upstream from the site of cleavage for the corresponding growth factor, with a substitution codon encoding a basic residue.

12. The polynucleotide according to claim 7, wherein the substitution codon encodes serine.

13. The polynucleotide according to claim 7, wherein the corresponding neurotrophin is selected from the group consisting of NGF, NT-3 and BDNF.

14. The polynucleotide of SEQ.ID.No. 16.

15. A polynucleotide encoding a mutant pro-neurotrophin, wherein the polynucleotide differs in nucleotide sequence from wild-type by replacement of the codon encoding a target asparagine residue, at a position 4 amino acids upstream from the site of cleavage for the corresponding neurotrophin, with a substitution codon encoding a basic residue.

16. The polynucleotide according to claim 15, wherein the substitution codon encodes serine.

17. The polynucleotide according to claim 15, wherein the corresponding neurotrophin is NT-4/5.

18. A recombinant expression vector containing the polynucleotide of any of claims 11, 14 or 15.

19. A host cell containing the recombinant expression vector of of any of claims 11, 14 or 15.

20. A pharmaceutical composition comprising the recombinant expression vector of of any of claims 11, 14 or 15.

21. A pharmaceutical composition comprising the host cell of any of claims 11, 14 or 15.

22. A process for producing a mutant pro-neurotrophin for use in intracellular processing of a corresponding growth factor having improved secretion efficiency as compared to wild-type growth factor, the process comprising (a) synthesis of the mutant pro-neurotrophin encoding polynucleotide, wherein the polynucleotide differs in nucleotide sequence from wild-type by replacement of the codon encoding a target asparagine residue, at a position 8 amino acids upstream from the site of cleavage for the corresponding growth factor, with a substitution codon encoding a basic residue; and (b) causing the synthetic polynucleotide to express the pro-neurotrophin.

23. The process according to claim 22, wherein the polynucleotide of claims 11 or 14 is produced by step (a).

24. A process for producing a mutant pro-neurotrophin for use in intracellular processing of a corresponding growth factor having improved secretion efficiency as compared to wild-type growth factor, the process comprising (a) synthesis of the mutant pro-neurotrophin encoding polynucleotide, wherein the polynucleotide differs in nucleotide sequence from wild-type by replacement of the codon encoding a target asparagine residue, at a position 4 amino acids upstream from the site of cleavage for the corresponding growth factor, with a substitution codon encoding a basic residue; and (b) causing the synthetic polynucleotide to express the pro-neurotrophin.

25. The process according to claim 22, wherein the polynucleotide of claim 15 is produced by step (a).
